(19) United States
(12) Patent Application Publication
(10) Pub. No.: US 2020/0330652 A1
(43) Pub. Date: Oct. 22, 2020

(54) WOUND TREATMENT DEVICE

(71) Applicant: LifeCell Corporation, Branchburg, NJ (US)

(72) Inventor: Israel Jessop, Annandale, NJ (US)

(21) Appl. No.: 16/060,143

(22) PCT Filed: Dec. 6, 2016
(86) PCT No.: PCT/US2016/065088

§ 371 Date: Jun. 7, 2018

Related U.S. Application Data
(60) Provisional application No. 62/266,249, filed on Dec. 11, 2015.

(51) Int. Cl. A61L 31/00

(52) U.S. Cl. CPC A61L 31/005; A61M 27/00; A61L 2430/22; A61L 2430/34

(57) ABSTRACT

The present disclosure provides devices for treating deep or tunneling wounds, such as fistulas. The device can include a regenerative tissue matrix that can be tailored to a variety of different wound shapes and sizes, and can be secured in place to help regenerate tissue within the wound.

This application claims priority to U.S. Provisional Patent Application No. 62/266,249, which was filed on Dec. 11, 2015 and which is incorporated by reference in its entirety.

The present disclosure relates generally to devices and methods for treating wounds, and in particular, to devices and methods for treating deep or tunneling wounds, including fistulas.

Deep wounds, including fistulas and tunneling wounds, can be challenging to treat and may require use of specialized fillers or plugs, including synthetic plugs and injectable collagen-based materials. Synthetic devices, although effective, do not regenerate natural tissue and require long-term presence of a foreign body. And injectable materials may become prematurely resorbed or may migrate from the target treatment site.

Accordingly, the present application provides improved devices and methods for treatment of deep, tunneling wounds, and/or fistulas. The improved devices allow regeneration of tissue at desired treatment sites, resist migration, and can be tailored to a variety of shapes and sizes to completely fill a wound or fistula space.

In certain aspects, a wound treatment device is provided. The device can include an elongated central body portion formed of a tissue matrix having a length and a width and a longitudinal axis extending along the length. The device can also include a group of tissue matrix bodies, wherein each of the tissue matrix bodies has a width that is wider than the width of the elongated central body portion, and wherein each of the tissue matrix bodies includes an opening passing there through such that a portion of the elongated central body portion can be passed through each of the bodies to form an elongated structure having a central support formed of the elongated central body portion, and space filling protrusions formed by the group of tissue matrix bodies.

In another aspect, a method of producing a wound treatment device is provided. The method can include selecting an elongated body portion formed of a tissue matrix having a length and a width and a longitudinal axis extending along the length and selecting a group of tissue matrix bodies, wherein each of the tissue matrix bodies has a width that is wider than the width of the elongated body portion, and wherein each of the tissue matrix bodies includes an opening passing there through. The method can further include passing a portion of the elongated body portion through the opening in each of the each of the tissue matrix bodies to form an elongated structure having a central support formed of the elongated body portion, and space filling protrusions formed by the group of tissue matrix bodies.

Also provided is a method of treating a wound. The method can include selecting a treatment device comprising an elongated central body portion formed of a tissue matrix having a length and a width and a longitudinal axis extending along the length; and a group of tissue matrix bodies, wherein each of the tissue matrix bodies have a width that is wider than the width of the elongated body portion, and wherein each of the tissue matrix bodies includes an opening passing therethrough, wherein a portion of the elongated central body portion passes through each of the tissue matrix bodies to form an elongated structure having a central support formed of the elongated central body, and space filling protrusions formed by the group of tissue matrix bodies. The method can further include passing an end portion of the elongated body portion through and opening in a wound and moving the elongated body portion into the opening until the space filling protrusions are positioned in a desired location within the wound.

Also provided are other wound treatment devices. The device can comprise an elongated central body portion formed of a tissue matrix having a length and a width and a longitudinal axis extending along the length. The devices can also comprise a compressible tissue matrix composition securely attached to the elongated central body, wherein the tissue matrix composition can be compressed to allow passage of the elongated central body portion and tissue matrix composition through a narrow opening.

Also provided are methods of treating a wound. The methods can comprise selecting a treatment device comprising an elongated central body portion formed of a tissue matrix having a length and a width and a longitudinal axis extending along the length; and a compressible tissue matrix composition securely attached to the elongated central body, wherein the tissue matrix composition can be compressed to allow passage of the elongated central body portion and tissue matrix composition through a narrow opening. The method can include passing an end portion of the elongated central body portion through and opening in a wound, and moving the elongated central body portion and compressible tissue matrix composition into the opening until the space filling protrusions are positioned in a desired location within the wound.

### BRIEF DESCRIPTION OF THE DRAWINGS

Reference will now be made to exemplary embodiments, examples of which are illustrated in the accompanying drawings. Wherever possible, the same reference numbers will be used throughout the drawings to refer to the same or like parts. The drawings are not necessarily to scale.

FIG. 1 is a perspective view of a device for treating deep, tunneling wounds and/or fistulas, according to certain embodiments.

FIG. 2 is an exploded view of the device of FIG. 1, prior to assembly.

FIG. 3. is a perspective view of a device for treating deep, tunneling wounds and/or fistulas, according to certain embodiments.

FIG. 4A is a side view of a device for treating deep, tunneling wounds and/or fistulas, according to certain embodiments prior to assembly.

FIG. 4B illustrates a method for assembling the device of FIG. 4A

FIG. 5 illustrates a method for implantation of a device for treating a tunneling wound on a lower limb.

FIG. 6 illustrates a method for treating an anal fistula using devices of the present application.

FIG. 7 illustrates another embodiment of a device for treating deep, tunneling wounds, and/or fistulas, including a drainage tube integrated with the device.

FIG. 8 illustrates a method of treatment using the device and drainage tube of FIG. 7.

FIG. 9A is a perspective view of a treatment device, according to certain embodiment.

FIG. 9B is a perspective view of the treatment device of FIG. 9A in an expanded configuration, according to certain embodiments.

FIG. 10 is a perspective view of a treatment device, according to certain embodiments.

FIG. 11 illustrates a method for treating an anal fistula using devices of the present application.

### DESCRIPTION OF EXEMPLARY EMBODIMENTS

Reference will now be made in detail to various embodiments of the disclosed devices and methods, examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Any range described herein will be understood to include the endpoints and all values between the endpoints.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

The present disclosure relates generally to devices and methods for treating deep or tunneling wounds. Such wounds can include, for example, tunneling wounds that form on the skin (e.g., on the limbs), and can extend through subcutaneous tissues, e.g., through fascia, muscle, and/or into bone. Such wounds can be associated with trauma, surgery, infection, and/or a variety of different diseases (e.g., vascular disorders and/or diabetes). In addition, for purposes of the present application, deep or tunneling “wounds” will be understood to include fistulas or other anatomic/structural malformations, including anal fistulas, recto-anal fistulas, fistulas relating to urinary structures, and any other abnormal anatomic openings or spaces that would desirably be closed by surgical or nonsurgical means.

The devices described herein can provide a number of improvements over existing materials used to repair or treat tunneling wounds and fistulas. For example, the devices described herein can be formed of regenerative materials, e.g., regenerative acellular tissue matrices that support the ingrowth of surrounding cells and regeneration of tissue. In some cases, the materials are selected to allow formation of tissue that is similar to naturally occurring tissue and has limited or no scar formation.

The devices described herein are formed such that a surgeon can tailor their size and shape to a particular wound. For example, the devices described herein are configured such that surgeons can easily tailor the shape and size, including the length, width, and curvature to completely or nearly completely fill a wound, including filling of long or tortuous wounds, such us fistulas.

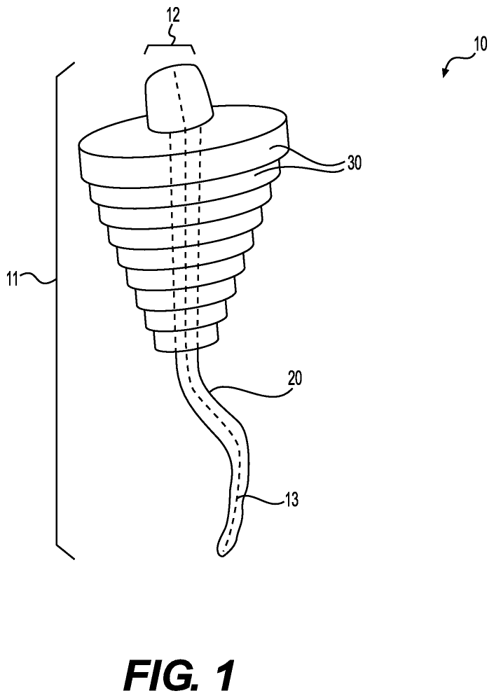

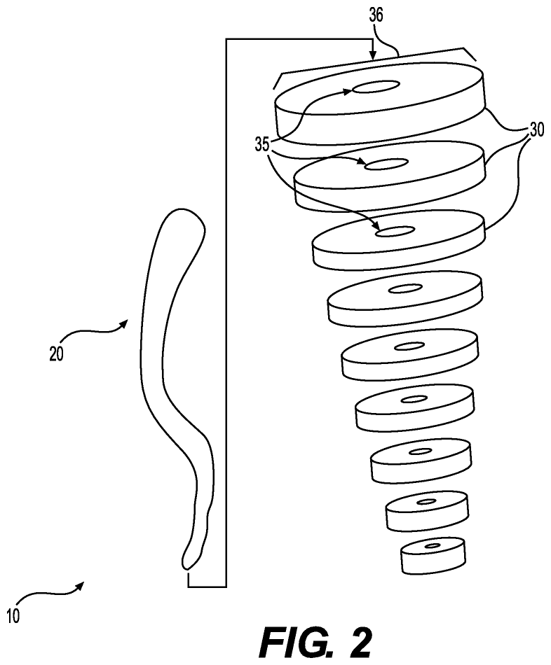

The devices and methods discussed herein can have a variety of configurations. For example, suitable exemplary devices are illustrated in FIGS. 1-4B. As shown in FIG. 1, the device 10 for treating deep, tunneling wounds and/or fistulas can include an elongated central body portion 20 formed of a tissue matrix having a length 11 and a width 12 and a longitudinal axis 13 extending along the length 12. In addition, the device 10 can include a group of widened sections 30, formed of tissue matrix bodies.

The elongated central body portion 20 can be formed of a flexible material such that the central body portion 20 can be passed into a curved or tortuous wound or fistula. For example, as noted above, the elongated central body portion 20 can be formed from an acellular tissue matrix, including, for example, a dermal acellular tissue matrix.

The term “acellular tissue matrix,” as used herein, refers generally to any tissue matrix that is substantially free of cells and/or cellular components. Skin, parts of skin (e.g., dermis), and other tissues such as blood vessels, heart valves, fascia, cartilage, bone, and nerve connective tissue may be used to create acellular matrices within the scope of the present disclosure. Suitable acellular tissue matrices can include ALLODERM® or STRATTICE™, which are human and porcine acellular dermal matrices, respectively (LIFECELL® CORPORATION). Alternatively, other suitable acellular tissue matrices can be used such as tissue matrices produced from tendon, ligament, vascular tissue, neural tissue, muscle tissue, intestinal tissue, small intestine submucosal tissue, bladder tissue, hepatic tissue, lung tissue, or dural tissue. For example, a number of suitable biological scaffold materials that may be used are described by Badylak et al., “Extracellular Matrix as a Biological Scaffold Material: Structure and Function,” Acta Biomaterialia (2008), doi:10.1016/j.actbio.2008.09.013.

The elongated central body portion 20 and tissue matrix bodies 30 can be configured to be connected to one another to produce a structure that can fill wounds of various sizes and dimensions. For example, as shown, each of the tissue matrix bodies 30 can include an opening 35 passing through the body 30 such that the a portion of the elongated central body portion 20 can be passed through each of the bodies 30 to form an elongated structure having a central support formed of the elongated central body portion 20, and space filling protrusions formed by the group of tissue matrix bodies 30.

The sizes of the tissue matrix bodies 30 can be selected to produce a space filling region that will completely or nearly completely fill a wound or fistula. For example, as shown, each of the tissue matrix bodies 30 has a width 36 that is wider than the width of the elongated central body portion 20. Furthermore, the width 36 of each of the bodies 30 can vary or be identical. For example, as shown the tissue matrix bodies 30 have decreasing widths along the length 11 of the central body portion 20. As such, the bodies 30 form a cone-shaped implant region to fill a tunneling wound.

The elongated central body portion 20 can also have a width 12 that varies along its length. For example, in one embodiment, the central body 20 has a width 11 that decreases from one end to another. As such, the central body 20 can be placed through the openings 35 in the tissue matrix bodies 30 and pulled through the opening until a widened end firmly abuts the edges of the openings 35, thereby securing the bodies 30 in place around the elongated central body portion 20.

As noted, each of the elongated central body 20 and group of tissue matrix bodies 30 can be produced from tissue matrix products. For example, in one embodiment, the entire device 10 is produced from acellular tissue matrices. Suitable tissue matrices can include, dermal tissue matrices tissue matrices derived from tendon, ligament, vascular tissue, neural tissue, muscle tissue, intestinal tissue, small intestine submucosal tissue, bladder tissue, hepatic tissue, lung tissue, or dural tissue.

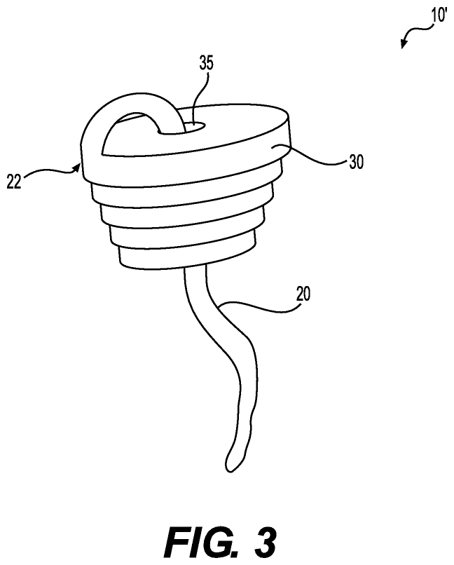

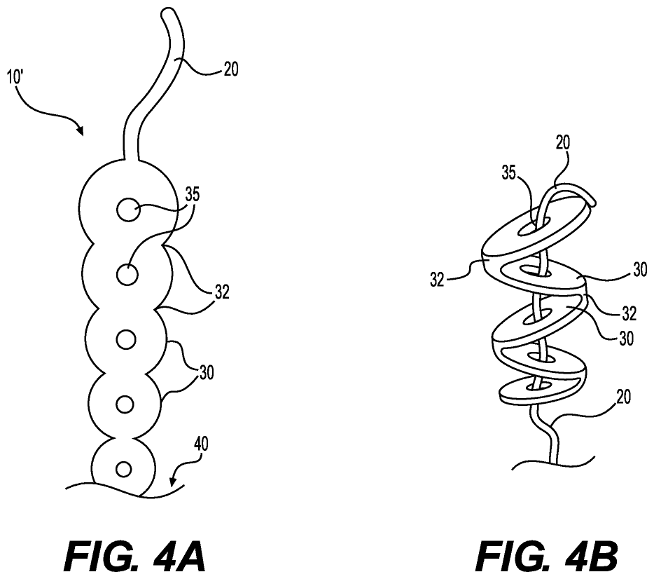

In one embodiment, the elongated central body portion 20 and group of tissue matrix bodies 30 are joined to one another. For example, FIG. 3. is a perspective view of a device 10′ for treating deep, tunneling wounds and/or fistulas, according to certain embodiment. And FIG. 4A is a side view of the device of FIG. 3 prior to assembly. As shown, the central body portion 20 and bodies 30 are produced from a single sheet of material, such as a sheet of acellular tissue matrix, wherein each of the tissue matrix bodies 30 are joined to one another at a connecting region 32, and one of the tissue matrix bodies is joined to the elongated central body portion 20.

Prior to use, a surgeon can assemble and size the device 10′. For example, FIG. 4B illustrates a method for assembling the device of FIG. 4A. As shown, the elongated central body portion 20 is passed through openings 35 in each of the tissue matrix bodies 30, which are folded at the connecting regions 32, thereby forming a chain-like configuration. The elongated central body portion 20 is then pulled to produce an appropriate amount of tension and optionally cut to a desired length. After proper placement, the device can be anchored, e.g., but using sutures, clips, or other suitable surgical anchor devices.

The devices 10, 10′ can have a variety of sizes, which can be adjusted by a surgeon prior to or during use. For example, the device 10′, of FIG. 4A has five tissue matrix bodies 30. However, the device 10′ can include additional bodies 30, connected at a distal end 40. During use, the surgeon may cut off unneeded bodies to produce a device of sufficient size for a given wound. Furthermore, additional free bodies 30, as shown in FIG. 1, can be added if needed.

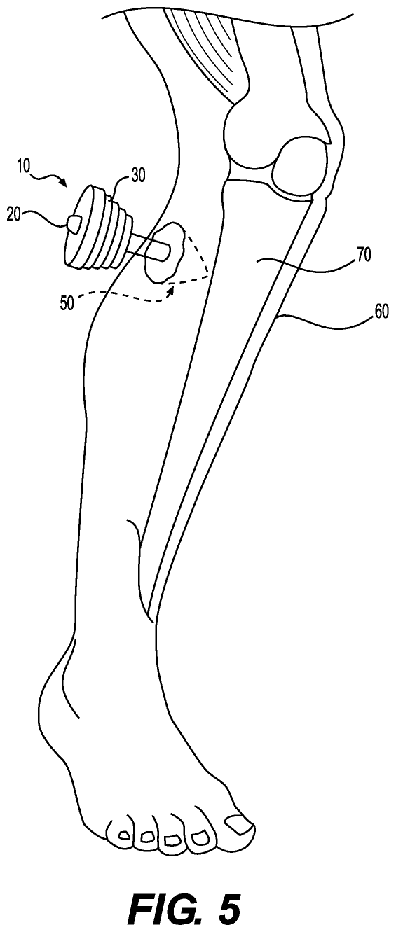

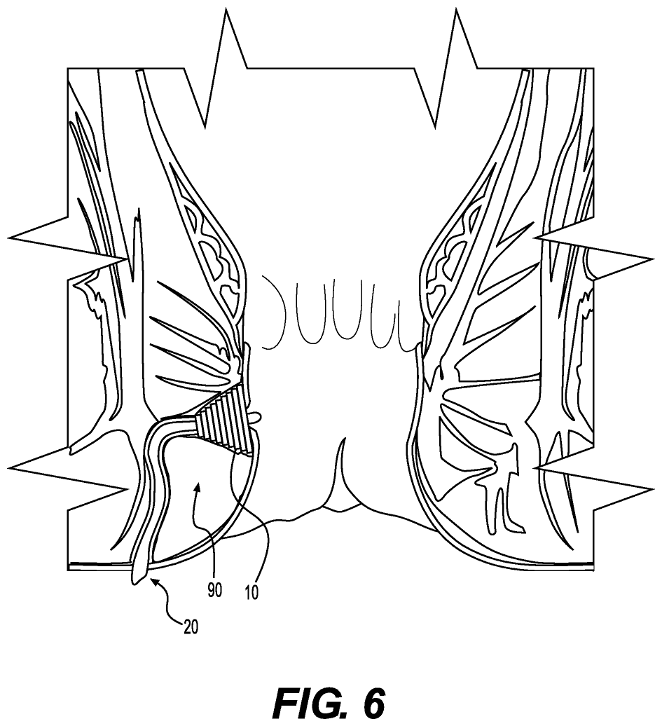

As discussed above, the present devices can be used for treatment of a variety of different types of wounds. For example, FIG. 5 illustrates a method for implantation of a device 10 for treating a tunneling wound 50 on a lower limb 60. Such tunneling wounds may start with cutaneous tissue and pass towards underlying structures such as bone 70. And FIG. 6 illustrates a method for treating an anal fistula 90 using devices 10 of the present application. As shown, the device 10 can be placed into the fistula 90 with the elongated central body portion 20 passing into the fistula towards the skin, thereby filling the fistula.

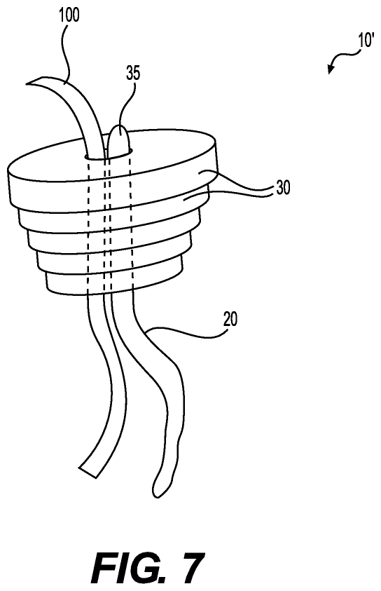

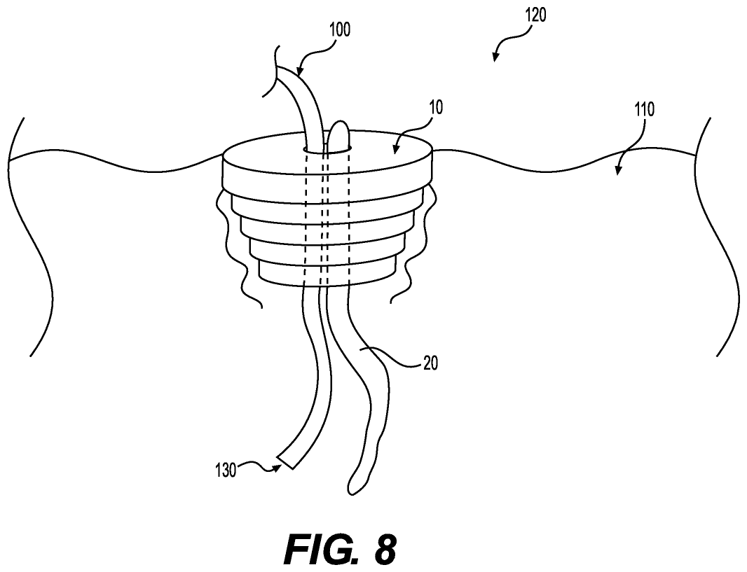

In some cases, the devices 10, 10′ may be used in conjunction with other wound treatment apparatuses. For example, FIG. 7 illustrates another embodiment of a device 10 for treating deep, tunneling wounds, and/or fistulas, including a drainage tube 100 integrated with the device, and FIG. 8 illustrates a method of treatment using the device 10 and drainage tube 100 of FIG. 7. for treating a wound 120 in skin 110 and/or subcutaneous tissue. As shown, the drainage tube 100 can be passed through the openings 35 such that a distal region 130 projects into the wound, thereby allowing deep drainage. In some cases, the drainage tube can include multiple openings along its length, thereby permitting suction within the device 10.

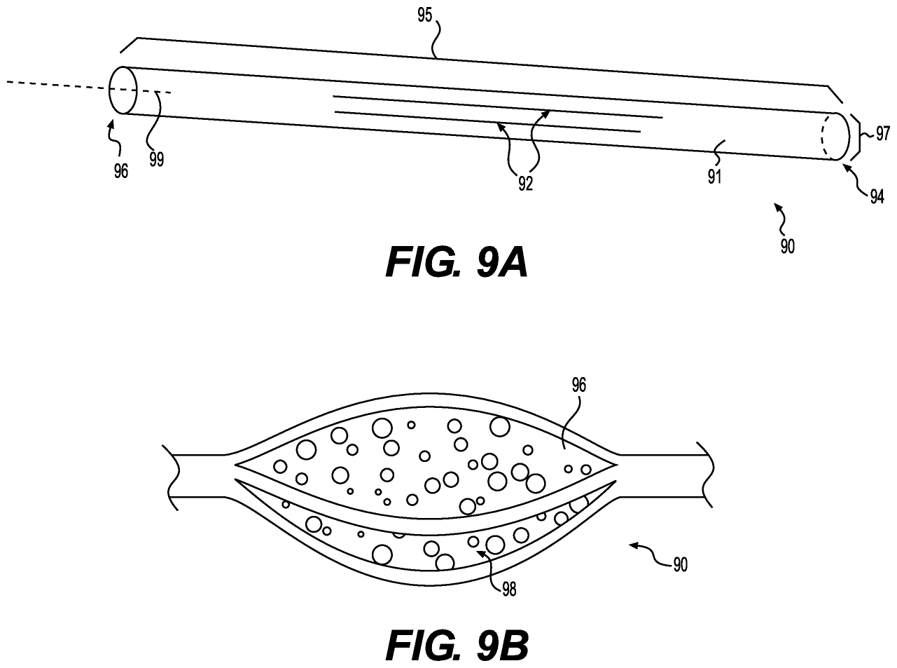

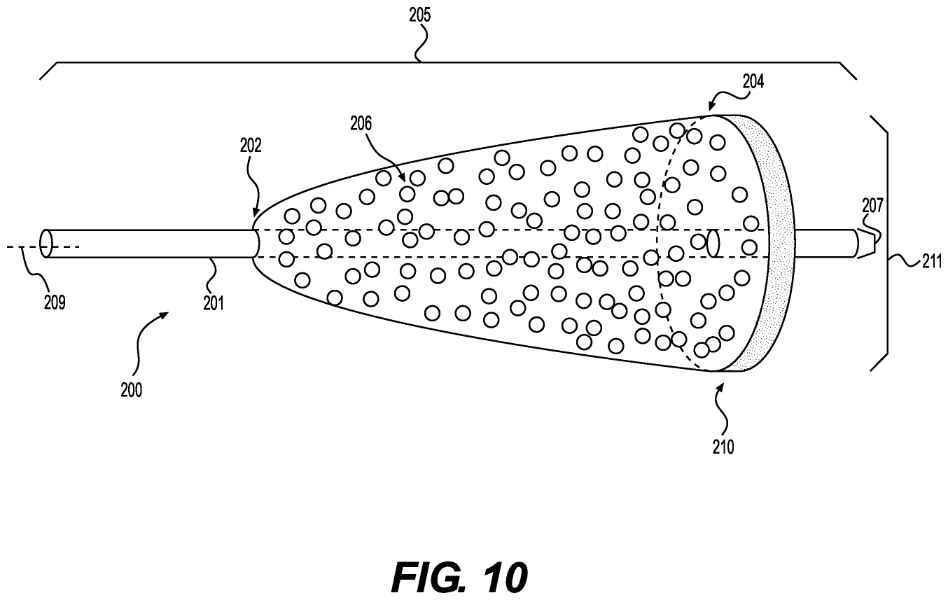

The devices disclosed herein can also be used to treat fistulas or other anatomic defects that have a widened portion over part or all of their lengths. For Example, FIG. 9A is a perspective view of a treatment device 90, according to certain embodiment, and FIG. 9B is a perspective view of the treatment device 90 of FIG. 9A in an expanded configuration, according to certain embodiments. In addition, FIG. 10 is a perspective view of a treatment device 200, according to certain embodiments.

The devices can comprise an elongated central body portion 91, 201 formed of a tissue matrix having a length 95, 205 and a width 97, 207 and a longitudinal axis 99, 209 extending along the length 95, 205. The devices can also comprise a compressible tissue matrix composition 96, 206 securely attached to the elongated central body 91, 201 wherein the tissue matrix composition 96, 206 can be compressed to allow passage of the elongated central body portion 91, 201 and tissue matrix composition 96, 206 through a narrow opening, such as an opening in a fistula.

The tissue matrix composition 96, 206 can be secured to the devices in a number of ways. For example, in the embodiment of FIGS. 9A and 9B, the central body portion 91 can include one or more openings 92 or slits, that form a central opening 98 in which the tissue matrix composition 96 can be contained in a manner that provides surface contact with the composition 96, upon implantation.

Alternatively, as shown in FIG. 10 the device 200 can include a composition 206 that is passed over the central body 201. Further, as shown in FIG. 10, the composition 206 can have a diameter 211 that increases from one end 202 to another 204, including, for example, a cone shape. Furthermore, one end can include a tissue matrix body 208 secured to the device 200 to assist in holding the composition in place.

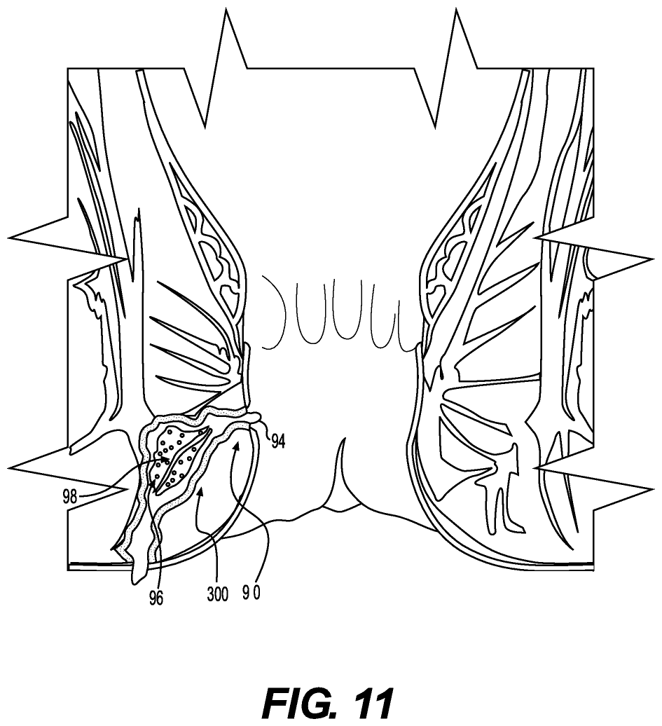

As noted above, the device 90, 200 can be used to treat fistulas or other defects having a widened portion. For example, FIG. 11 illustrates a method for treating an anal fistula 300 using devices 90 of the present application. As shown, one end 94, 96, of the device 90 is passed through an opening 310 of the fistula 300, and the tissue matrix composition 96 is allowed to expand to fill a cavernous or widened portion 320, thereby substantially filling the defect and allowing proper treatment.

The tissue composition 96, 206 of FIGS. 9A-11 can be produced from a variety of materials, but as noted, suitable materials should be compressible such that the material can be passed into a narrow opening and expand to fill a larger or wider area in a fistula or other defects. Accordingly, in some embodiments, the tissue composition, can include a sponge or similar material. A tissue sponge, can include, for example, any tissue matrix material that has been cut or micronized to produce a tissue matrix suspension, and resuspended to form a sponge-like material.

Suitable sponges can be produced from materials such as dermal or adipose tissue, as described for example, in US Patent Publication Numbers 2012/0310367 A1 to Connor, 2010/0040687 to Pedrozo et al., and 2012/0263763 A1 to Sun et al.

It should be noted that various therapeutic agents can be incorporated into the devices disclosed herein. For example, in various embodiments, the devices can include one or more antimicrobials (antibiotics, antivirals, antifungals), thrombotic agents, chemotherapeutic agents, or growth factors.

Other embodiments will be apparent to those skilled in the art from consideration of the specification and practice of this disclosure. It is intended that the specification and examples be considered as exemplary only, with the true scope and spirit of the disclosed devices and methods being indicated by the following claims.

1. A wound treatment device, comprising:
an elongated central body portion formed of a tissue matrix having a length and a width and a longitudinal axis extending along the length;
a group of tissue matrix bodies, wherein each of the tissue matrix bodies has a width that is wider than the width of the elongated central body portion, and wherein each of the tissue matrix bodies includes an opening passing there through, such that a portion of the elongated central body portion can be passed through each of the bodies to form an elongated structure having a central support formed of the elongated central body portion, and space filling protrusions formed by the group of tissue matrix bodies.

2. The device of claim 1, wherein the elongated central body portion is formed of an acellular tissue matrix.

3. (canceled)

4. The device of claim 1, wherein the elongated central body portion comprises a tissue matrix derived from tissue selected from tendon, ligament, vascular tissue, neural tissue, muscle tissue, intestinal tissue, small intestine submucosal tissue, bladder tissue, hepatic tissue, lung tissue, or dural tissue.

5. The device of claim 1, wherein each of the tissue matrix bodies is formed of an acellular tissue matrix.

6. (canceled)

7. The device of claim 1, wherein each of the tissue matrix bodies comprises a tissue matrix derived from tissue selected from tendon, ligament, vascular tissue, neural tissue, muscle tissue, intestinal tissue, small intestine submucosal tissue, bladder tissue, hepatic tissue, lung tissue, or dural tissue.

8. The device of claim 1, wherein the elongated central body portion and each of the tissue matrix bodies are joined to one another.

9. The device of claim 8, wherein the elongated central body portion and each of the tissue matrix bodies are formed of a single piece of material.

10. The device of claim 1, wherein the width of the elongated central body portion varies along the length of the elongated central body portion.

11. The device of claim 10, wherein the width of the elongated central body portion decreases from one end to a second end.

12. The device of claim 1, wherein the widths of the tissue matrix bodies vary.

13. The device of claim 12, wherein the tissue matrix bodies are connected to one another in a chain configuration, and wherein the width of the tissue matrix bodies decreases along a length of the chain configuration.

14. The device of claim 1, wherein the elongated central body portion has been passed through the opening in each of the tissue matrix bodies to form an elongated structure having a central load-bearing support and tissue matrix protrusions for treating a wound.

15-28. (canceled)

29. A method of treating a wound, comprising:
selecting a treatment device comprising:
an elongated central body portion formed of a tissue matrix having a length and a width and a longitudinal axis extending along the length; and
a group of tissue matrix bodies, wherein each of the tissue matrix bodies has a width that is wider than the width of the elongated central body portion, and wherein each of the tissue matrix bodies includes an opening passing there through, wherein a portion of the elongated central body portion passes through each of the tissue matrix bodies to form an elongated structure having a central support formed of the elongated central body portion, and space filling protrusions formed by the group of tissue matrix bodies; and
passing an end portion of the elongated central body portion through an opening in a wound, and moving the elongated central body portion into the opening until the space filling protrusions are positioned in a desired location within the wound.

30. The method of claim 29, wherein the wound comprises one or more of a tunneling wound, a fistula, and an anal fistula.

31-32. (canceled)

33. The method of claim 29, wherein the elongated central body portion is formed of an acellular tissue matrix.

34. (canceled)

35. The method of claim 29, wherein the elongated central body portion comprises a tissue matrix derived from tissue selected from tendon, ligament, vascular tissue, neural tissue, muscle tissue, intestinal tissue, small intestine submucosal tissue, bladder tissue, hepatic tissue, lung tissue, or dural tissue.

36. The method of claim 29, wherein each of the tissue matrix bodies is formed of an acellular tissue matrix.

37. (canceled)

38. The method of claim 29, wherein each of the tissue matrix bodies comprises a tissue matrix derived from tissue selected from tendon, ligament, vascular tissue, neural tissue, muscle tissue, intestinal tissue, small intestine submucosal tissue, bladder tissue, hepatic tissue, lung tissue, or dural tissue.

39. The method of claim 29, wherein the elongated central body portion and each of the tissue matrix bodies are joined to one another.

40. The method of claim 39, wherein the elongated central body portion and each of the tissue matrix bodies are formed of a single piece of material.

41. The method of claim 29, wherein the width of the elongated central body portion varies along the length of the elongated central body portion.

42. The method of claim 41, wherein the width of the elongated central body portion decreases from one end to a second end.

43. The method of claim 29, wherein the widths of the tissue matrix bodies vary.

44. The method of claim 43, wherein the tissue matrix bodies are connected to one another in a chain configuration, and wherein the width of the tissue matrix bodies decreases along a length of the chain configuration.

45-68. (canceled)
